SEQUENCE LISTING (14 sequences)

SEQ ID NO: 1 - protein, 775 aa, Thermococcus sp. 9N-7
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFAIATLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRLIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 2 - DNA, 20 bp, Artificial Sequence
  synthetic
gacactcgtatgcagtagcc

SEQ ID NO: 3 - DNA, 40 bp, Artificial Sequence
  synthetic
cttttaagaaccggacgaacgacactcgtttgcagtagcc

SEQ ID NO: 4 - DNA, 20 bp, Artificial Sequence
  synthetic
tgtctacacgcaagcttaca

SEQ ID NO: 5 - DNA, 20 bp, Artificial Sequence
  synthetic
cttttaagaaccggacgaac

SEQ ID NO: 6 - DNA, 100 bp, Artificial Sequence
  synthetic
tgtctacacgcaagcttacaccattctttaacagtatcactatatccatttacgagtcaacattaacctcggctactgcatacgagtgtcaaaaaaaaaa

SEQ ID NO: 7 - DNA, 100 bp, Artificial Sequence
  synthetic
tgtctacacgcaagcttacattaagactcgccatgttacgatctgccaagtacagccttgaatcgtcactggctactgcatacgagtgtcaaaaaaaaaa

SEQ ID NO: 8 - DNA, 100 bp, Artificial Sequence
  synthetic
tgtctacacgcaagcttacattaagactcgacatgatacgatctgacaagaacagacttgaatcgacactggctactgcatacgagtgtcaaaaaaaaaa

SEQ ID NO: 9 - DNA, 100 bp, Artificial Sequence
  synthetic
tgtctacacgcaagcttacaaactccattacctattcaacttacaatcctatcaaccttataatccacttggctactgcatacgagtgtcaaaaaaaaaa

SEQ ID NO: 10 - RNA, 16 nt, Artificial Sequence
  synthetic
aaaauuuauuuauuaa

SEQ ID NO: 11 - DNA, 16 bp, Artificial Sequence
  synthetic
aaaatttatttattaa

SEQ ID NO: 12 - DNA, 16 bp, Artificial Sequence
  synthetic
aaaatttatttattaa

SEQ ID NO: 13 - RNA, 70 nt, Artificial Sequence
  synthetic
gggaggaggauuaccccucguuaauaaauaaauuuucucucgugaucggguagcuggacgcgacgggucc

SEQ ID NO: 14 - DNA, 16 bp, Artificial Sequence
  synthetic
aaaatttatttattaa

(19) United States
(12) Patent Application Publication
(10) Pub. No.: US 2016/0138063 A1
(43) Pub. Date: May 19, 2016

(54) METHDS AND COMPOSITIONS FOR REPLICATION OF THREOSE NUCLEIC ACIDS

(71) Applicants: John Chaput, Phoenix, AZ (US); Hanyang Yu, New Haven, CT (US); Su Zhang, Houston, TX (US); Matthew R. Dunn, Scottsdale, AZ (US)

(72) Inventors: John Chaput, Phoenix, AZ (US); Hanyang Yu, New Haven, CT (US); Su Zhang, Houston, TX (US); Matthew R. Dunn, Scottsdale, AZ (US)

(21) Appl. No.: 14/758,844

(22) PCT Filed: Jan. 2, 2014
(86) PCT No.: PCT/US2014/010062

§ 371 Date: Jul. 1, 2015

Related U.S. Application Data
(60) Provisional application No. 61/748,834, filed on Jan. 4, 2013.

(51) Int. Cl. C12P 19/34; C12N 15/10

(52) U.S. Cl. CPC C12P 19/34; C12N 15/1058; C12N 15/1093

(57) ABSTRACT

Methods and compositions for replication of threose nucleic acids (TNAs) are described. The described methods include a method for transcribing a DNA template into a TNA, and a method for reverse transcribing a threose nucleic acid into a cDNA.

### CROSS-REFERENCE TO RELATED APPLICATION

This application claims priority to U.S. provisional patent application 61/748,834 filed on Jan. 4, 2013, which incorporated by reference herein in its entirety.

### STATEMENT AS TO RIGHTS TO INVENTIONS MADE UNDER FEDERALLY SPONSORED RESEARCH AND DEVELOPMENT

Not applicable.

### BACKGROUND

The emerging field of synthetic genetics provides an exciting opportunity to explore the structural and functional properties of synthetic genetic polymers by in vitro selection. However, achieving the goal of artificial genetics requires the ability to synthesize unnatural nucleic acid substrates (“XNA”s), such as threose-nucleic acids (“TNAs”), that are not otherwise available. Limiting this process is the availability of enzymes and conditions that allow for the storage and propagation of genetic information present in unnatural nucleic acid polymers such as TNAs.

### BRIEF SUMMARY

Described herein are methods, compositions, and systems for replicating and evolving threose nucleic acids.

Accordingly, in a first aspect disclosed herein is a method for synthesizing a threose nucleic acid, comprising: contacting a single stranded DNA template hybridized to a primer with a DNA polymerase comprising an amino acid sequence at least 95% identical to the amino acid sequence of SEQ ID NO:1 in the presence of tTTP, tGTP, tATP; and (i) dCTP; or (ii) a combination of tCTP and dCTP; and incubating at a temperature suitable for polymerization by the DNA polymerase to obtain a threose nucleic acid.

In some embodiments of the first aspect, the DNA polymerase is in the presence of tTTP, tGTP, tATP, and dCTP. In some embodiments, where the DNA polymerase is in the presence of tTTP, tGTP, tATP, and dCTP, the contacting step is done in the substantial absence of tCTP. In some embodiments, a threose nucleic acid is provided that is generated according to the just-mentioned method, where the synthesis reaction includes dCTP and is substantially free of tCTP.

In some embodiments of the first aspect, the DNA polymerase is in the presence of tATP, tTTP, tGTP, and a combination of tCTP and dCTP.

In some embodiments of the first aspect, the DNA polymerase comprises the amino acid sequence of SEQ ID NO:1.

In some embodiments of the first aspect, the single stranded DNA template sequence is restricted to the nucleotides dA, dC, and dT.

In other embodiments of the first aspect, the single stranded DNA template sequence comprises 7-deaza-dGTP instead of dGTP.

In a second aspect disclosed herein is a method for reverse transcribing a threose nucleic acid, comprising contacting a threose nucleic acid template with a SuperScript II reverse transcriptase in the presence of a primer and dNTPs, dNTP analogs, or a combination thereof to obtain a threose nucleic acid reverse-transcription mix, and incubating the mix at a temperature suitable for SuperScript II reverse transcriptase activity to obtain a cDNA copy of the threose nucleic acid template, where the threose nucleic acid template comprises deoxycytidine.

In a third aspect disclosed herein is a for molecular evolution of threose nucleic acids, where the method includes the steps of: (i) providing a DNA template library comprising diverse DNA template sequences; (ii) hybridizing the template library with one or more complementary primer sequences; (iii) incubating the hybridized template library with a DNA polymerase comprising an amino acid sequence at least 95% identical to the amino acid sequence of SEQ ID NO:1 in the presence of tTTP, tGTP, tATP; and (i) dCTP; or (ii) a combination of tCTP and dCTP; and incubating at temperature suitable for polymerization by the DNA polymerase to obtain a cTNA library; (iv) subjecting the cTNA library to a selection assay to obtain at least one or more selected cTNAs; and (v) incubating the one or more selected cTNAs with a primer, a SuperScript II reverse transcriptase, and dNTPs at a temperature suitable for SuperScript II reverse transcriptase activity to obtain to obtain a selected DNA template library.

In some embodiments of the third aspect, the diverse DNA template sequences are restricted to the nucleotides dA, dC, and dT.

In some embodiments of the third aspect, the selection assay in step (iv) comprises selection of one or more cTNAs based on affinity for a ligand. In some embodiments the selection assay in step (iv) comprises selection of one or more cTNAs based on a catalytic activity. In other embodiments the selection assay in step (iv) comprises selection of one or more cTNAs based on fluorescence emission.

In some embodiments of the third aspect, step (iii) is done in the substantial absence of tCTP.

In some embodiments of the third aspect, the DNA template library comprises DNA templates comprising 7-deaza-dGTP instead of dGTP.

In a fourth aspect disclosed herein is a TNA transcription system comprising a single stranded DNA template, a DNA polymerase comprising an amino acid sequence at least 95% identical to the amino acid sequence of SEQ ID NO:1, tTTP, tGTP, tATP; and (i) dCTP; or (ii) a combination of tCTP and dCTP.

In some embodiments of the fourth aspect the TNA transcription system comprises dCTP, but is substantially free of tCTP.

In some embodiments of the fourth aspect, the single stranded DNA template comprises 7-deaza-dGTP instead of dGTP.

In a fifth aspect disclosed herein is a TNA reverse transcription system comprising a TNA template, a SuperScript II reverse transcriptase, and dNTPs; wherein the TNA template comprises dC.

### BRIEF DESCRIPTION OF THE DRAWINGS

The present invention will be better understood and features, aspects and advantages other than those set forth above will become apparent when consideration is given to the following detailed description thereof. Such detailed description makes reference to the following drawings, wherein:

FIG. 1 shows a schematic illustration of Darwinian evolution of natural and artificial nucleic acid polymers. (A) In vitro evolution of nucleic acid polymers requires polymerases and reaction conditions that permit transcription, reverse-transcription, and amplification of genetic information in the laboratory. Each cycle of in vitro selection and amplification includes transcription of a DNA library into XNA, isolation of XNA molecules with a desired function, reverse-transcription of functional molecules back into DNA, and amplification of the resulting cDNA molecules by the polymerase chain reaction (PCR). This process, which is similar to in vitro RNA replication, requires copying of genetic information back and forth between DNA and XNA. (B) Constitutional structures for the linearized backbones of ribonucleic acid (left) and α-L-threofuranosyl-(3′→2′) nucleic acid (TNA) (right). TNA has a backbone repeat unit that is one atom shorter than the backbone repeat unit found in RNA (and DNA).

FIG. 2 (A) Chemical structures of TNA triphosphates (tNTPs). Diaminopurine is an analogue of adenine that forms three hydrogen bonds with thymine. (B), Schematic representation of DNA primer extension reaction used to synthesize long TNA strands. The extended TNA product is shown as the top strand. (C), Therminator™ polymerase-mediated TNA transcription reactions analyzed by denaturing polyacrylamide gel electrophoresis. “A” refers to primer extension reactions performed using tATP in the reaction mixture, while “D” refers to primer extension reactions that contain tDTP in place of tATP.

FIG. 3 Enzyme-mediated reverse transcription of TNA into DNA. (A) Schematic representation of TNA-directed DNA polymerization. The TNA region is shown as the top strand, while the DNA region is shown as the bottom strand. (B), SuperScript™ II-mediated TNA reverse transcription reactions analyzed by denaturing polyacrylamide gel electrophoresis. Mn²⁺ is required to convert TNA into full-length DNA. (C), Time course analysis of DNA synthesis on TNA templates. Polymerization reactions were analyzed by denaturing polyacrylamide gel electrophoresis. The A and D templates refer to TNA templates containing either adenosine or diaminopurine in the TNA strand.

FIG. 4. Fidelity of TNA replication using a four-letter genetic alphabet. (A), Mutation profile of TNA replication indicates a high frequency of G->C substitutions. (B), Analysis of the local sequence context upstream and downstream of the misincorporation site demonstrates a sequence-specific context that favors mutagenesis when G residues are preceded by pyrimidines (C or T) in the DNA template. (C) Substituting tCTP in the reaction mixture for dCTP abolishes tGTP misincorporation opposite deoxyG in the template and reduces the error rate from 3.6×10⁻² to 3.5×10⁻³.

FIG. 5 An efficient and faithful replication system for TNA. (A) Replication of a three letter TNA library. An unbiased DNA library composed of three nucleotides (A, C and T) transcribes into TNA (left panel) and reverse transcribes back into DNA (right panel) with high primer-extension efficiency. (B) Mutation profile demonstrates that replication occurs with an error rate of 3.8×10⁻³ indicating a fidelity of 99.6%.

FIG. 6 TNA sensitivity to nuclease degradation. Nuclease stability of synthetic DNA, RNA, and TNA oligonucleotides was monitored over time by denaturing polyacrylamide gel electrophoresis. (A), In the presence of RQ1 DNase, DNA exhibits a half-life of ˜30 minutes, while TNA remains undigested after 72 hours. (B) In the presence of RNase A, RNA is digested in less that 5 seconds, while TNA remains completely intact after 72 hours. (C) RNase H digestion using DNA and TNA probes that are complementary in sequence to a longer RNA target indicates that TNA is not a substrate for RNase H. The gradient for the DNA probe was 0-30 minutes. The gradient for the TNA probe was 0-16 hours. The control was a no enzyme reaction over the same time period.

FIG. 7 TNA reverse transcription reactions were evaluated by challenging different enzymes to extend a DNA primer annealed to TNA template with dNTPs in the absence or presence of Mn²⁺. RT521 could incorporate several monomers under optimal conditions (100 nM primer-template complex, 1× ThermoPol buffer, 500 μM dNTPs, 1 mM MgSO₄ and 0.02 μg/μl RT521 enzyme). Incubation for 24 hours at 65° C. before pausing on the primer-template complex, and no discrete band for full-length product was observed. Also, manganese ions seemed to inhibit RT521's activity. SuperScript II reverse transcriptase could yield substantial amounts of full-length products in the presence of MnCl₂. TNA templates 1 and 2 were synthesized on DNA templates 4NT.8G and 4NT.3G, respectively. M: marker.

FIG. 8 Schematic of fidelity measurement. Primer P2 (Table 1) is designed to have a noncomplementary overhang on the 5′ end and an internal A:A mismatch reference position5. After the primer is extended with tNTPs, the chimeric DNA-TNA strand is separated from DNA template strand by denaturing PAGE. The purified TNA sequence is used as template in reverse transcription reaction, which generates full-length cDNA strand that is amplified by PCR. One of the PCR primer (P4) shares the same sequence as the 5′ overhang in P2 so that only full-length cDNA can be amplified even if some original DNA template contaminant is present. After PCR amplification, the internal reference position has a T residue and can be unambiguously distinguished from the original DNA template sequence.

FIG. 9 Amplification of cDNA after TNA reverse transcription. PCR amplification of cDNA generated in TNA reverse transcription was analyzed on 2% agarose gel. Cycle optimization showed exponential enrichment of cDNA sequences. N: negative control using purified TNA strand before reverse transcription as template. P: positive control using DNA library 3 NT.ATC as template. M: low DNA mass ladder.

FIG. 10 Fidelity of TNA replication under various transcription conditions. Table showing the fidelity of replication of DNA to TNA to DNA measured by sequencing the derived cDNA products under various transcription conditions, and calculating the single nucleotide misincorporation rate.

FIG. 11 TNA transcription on templates containing continuous GG regions. TNA transcription reactions on different DNA templates were analyzed on 20% denaturing polyacrylamide gel. TNA transcription on DNA templates containing multiple GG repeats (DNA template 4NT.10G.1 and 4NT.10G.2 on lane 2 and 3, respectively) (Table 1) generated truncated sequences with lower yield in full-length products, while DNA template devoid of G residues (3NT.ATC on lane 1) (Table 1) directed almost quantitative conversion of DNA primer to full-length DNA-TNA heteropolymer.

FIG. 12 Substitution profile and overall fidelity of TNA replication under different conditions. TNA replication were examined under different conditions where G:G mispair was disfavored, such as (a) using dGTP in place of tGTP during TNA transcription; (b), decreasing tGTP:tCTP ratio during TNA transcription; and (c), excluding tGTP during TNA transcription on a DNA template devoid of C (3NT.ATG in Table 1).

FIG. 13 An efficient and faithful replication system for transcribing unbiased four nucleotide libraries into TNA. An efficient and faithful replication system for transcribing unbiased four nucleotide libraries into TNA was established using DNA intermediates containing 7-deaza-G. DNA intermediates were generated by asymmetric PCR with 7-deaza-dGTP in place of dGTP. (A) Replication of a four-letter TNA library. An unbiased DNA library composed of four nucleotides (A, C, T and deaza-G) transcribes into TNA (left panel) and reverse transcribes back into DNA (right panel) with high primer-extension efficiency. (B) Mutation profile demonstrates that replication occurs with an error rate of 3.5×10⁻³ indicating a fidelity of 99.6%.

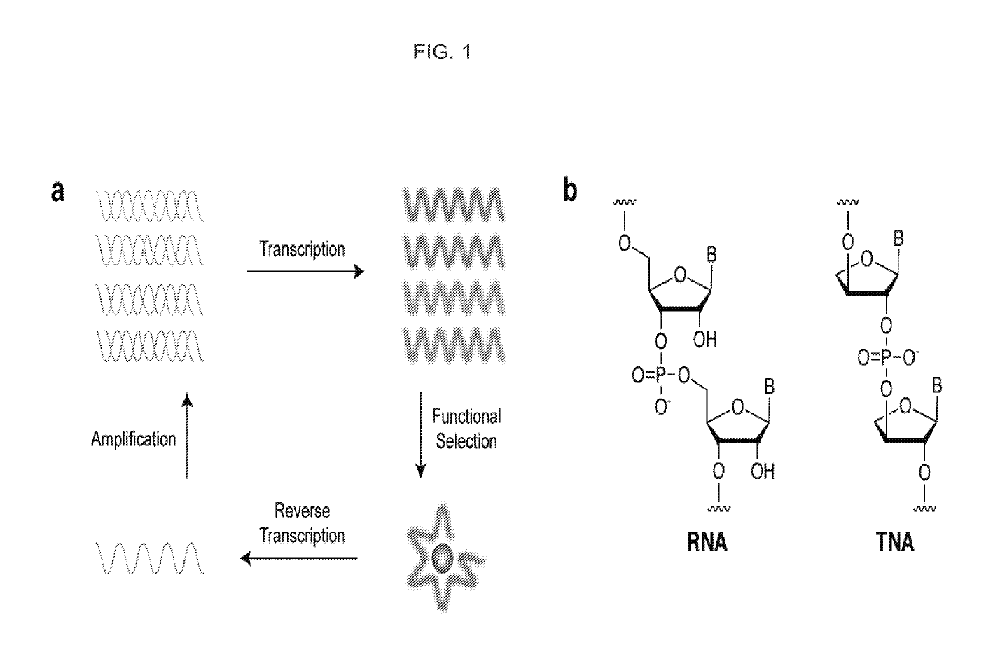

### DETAILED DESCRIPTION

Disclosed herein are methods, compositions and systems for replication and in vitro evolution of TNAs based on the unexpected finding that certain TNA synthesis conditions, as described herein, permit the efficient and faithful synthesis of XNAs from DNA templates and their reverse transcription into cDNAs using known polymerases.

Unless defined otherwise, all technical and scientific terms used herein have the same meaning as commonly understood by one of ordinary skill in the art to which the invention pertains. Although any methods and materials similar to or equivalent to those described herein can be used in the practice or testing of the present invention, the preferred methods and materials are described herein.

### DEFINITIONS

In describing the embodiments and claiming the invention, the following terminology will be used in accordance with the definitions set out below.

As used herein, “about” means within 5% of a stated range within the relevant parameter.

As used herein, “TNA” or “TNAs” refer to nucleic acids having a backbone composed primarily of α-L-threofuranosyl-(3′→2′) (threose)-containing nucleotides, but may include heteropolymers comprising both tNTPs and dNTPs (e.g., dC).

As used herein, “tNTPs” refer to threose nucleotide triphosphates.

As used herein, “tNTP analog” refers to a threose nucleotide triphosphate having a modified base moiety.

With respect to the amino acid sequence homology of polypeptides described herein, one of ordinary skill in the art will appreciate that structural and functional homology of two or polypeptides generally includes determining the percent identity of their amino acid sequences to each other. Sequence identity between two or more amino acid sequences is determined by conventional methods. See, for example, Altschul et al., (1997), Nucleic Acids Research, 25(17):3389-3402; and Henikoff and Henikoff (1982), Proc. Natl. Acad. Sci. USA, 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (ibid.). The percent identity is then calculated as: ([Total number of identical matches]/[length of the longer sequence plus the number of gaps introduced into the longer sequence in order to align the two sequences])(100).

Described herein are methods for efficient synthesis of a TNA from a DNA template. In various embodiment the methods include the steps of contacting a single stranded DNA template hybridized to a primer with a DNA polymerase comprising an amino acid sequence at least 95% (e.g., 97%, 98%, 99%, or 100%) identical to the amino acid sequence of Therminator™ DNA polymerase known under the tradename Therminator™ polymerase (New England Biolabs, MA) in the presence of tTTP, tGTP, tATP; and (i) dCTP; or (ii) a combination of tCTP and dCTP. The amino acid sequence of Therminator™ DNA polymerase is shown below as SEQ ID NO:1.

(SEQ ID NO: 1; amino acid sequence of Therminator ™
DNA polymerase)
MILDTDYITENGKPVIRVFKKENGEFKIEYDRTFEPYFYALLKDDSAIED
VKKVTAKRHGTVVKVKRAEKVQKKFLGRPIEVWKLYFNHPQDVPAIRDRI
RAHPAVVDIYEYDIPFAKRYLIDKGLIPMEGDEELTMLAFAIATLYHEGE
EFGTGPILMISYADGSEARVITWKKIDLPYVDVVSTEKEMIKRFLRVVRE
KDPDVLITYNGDNFDFAYLKKRCEELGIKFTLGRDGSEPKIQRMGDRFAV
EVKGRIHFDLYPVIRRTINLPTYTLEAVYEAVFGKPKEKVYAEEIAQAWE
SGEGLNELAPNERVARYSMEDAKVTYELGREFFPMEAQLSRLIGQSLWDV
SRSSTGNLVEWFLLRKAYKRKPDERELARRRGGYAGGYVKEPERGLWDNI
VYLDFRSLYPSIIITHNVSPDTLNREGCKEYDVAPEVGHKFCKDFPGFIP
SLLGDLLEERQKIKRKMKATVDPLEKKLLDYRQRLIKILANSFYGYYGYA
KARWYCKECAESVTAWGREYIEMVIRELEEKFGFKVLYADTDGLHATIPG
ADAETVKKKAKEFLKYINPKLPGLLELEYEGFYVRGFFVTKKKYAVIDEE
GKITTRGLEIVRRDWSEIAKETQARVLEAILKHGDVEEAVRIVKEVTEKL
SKYEVPPEKLVIHEQITRDLRDYKATGPHVAVAKRLAARGVKIRPGTVIS
YIVLKGSGRIGDRAIPADEFDPTKHRYDAEYYIENQVLPAVERILKAFGY
RKEDLRYQKTKQVGLGAWLK VKGKK

In some embodiments, the DNA polymerase comprises an A485L point mutation relative to the amino acid sequence of the 9° N DNA polymerase and is greater than about 95% identical to the amino acid sequence of Therminator™ DNA polymerase (Therminator™ DNA polymerase), e.g., about 96%, 97%, 98%, 99%, or 100% identical to the amino acid sequence of Therminator™ DNA polymerase. In one embodiment, the DNA polymerase to be used comprises the amino acid sequence of SEQ ID NO:1. Typically, TNA synthesis using the Therminator™ polymerase is carried out at about 50° C. to about 60° C. In some embodiments, the TNA synthesis reaction is carried out at about 55° C. Suitable concentrations of tNTPs range from about 20 μM to about 100 μM, e.g., about 25, 30, 35, 40, 50, 60, 70, 80, or another concentration of tNTPs from about 20 μM to about 100 μM.

In some embodiments, the single stranded DNA template to be used in the method comprises a sequence that is restricted to the nucleotides dA, dC, and dT. While not wishing to be bound by theory, it is believed that by limiting single stranded templates to sequences containing these three nucleotides, the fidelity of the sequence transcribed into TNAs is significantly increased as described herein. In other embodiments, the single stranded DNA template to be used comprises 7-deaza-dGTP instead of dGTP to reduce or eliminate dG-tG mispairing, and thereby increase replication fidelity. Also encompassed herein are heteropolymeric TNAs generated by the above-described method, which include tA, tT, tG, and dC.

Also described herein is method for reverse transcribing a TNA. In various embodiments, a TNA is reverse transcribed by a method that includes: contacting a TNA template that contains dCTP with a SuperScript II reverse transcriptase in the presence of a primer and dNTPs, and incubating the resulting mix, at a temperature suitable for SuperScript II reverse transcriptase activity, to obtain a cDNA copy of the TNA template.

Typically the reverse transcription reaction using the SuperScript II reverse transcriptase is carried out at a temperature of about 37° C. to about 45° C. In some embodiments, the TNA reverse transcription reaction is carried out at 42° C.

Also disclosed herein is a method for molecular evolution of threose nucleic acids, which includes the steps of: (i) providing a DNA template library containing diverse DNA template sequences; (ii) hybridizing the template library with one or more complementary primer sequences; (iii) incubating the hybridized template library with a DNA polymerase comprising an amino acid sequence at least 95% (e.g., 97%, 98%, 99%, or 100%) identical to the amino acid sequence of SEQ ID NO:1 in the presence of tTTP, tGTP, tATP, and dCTP, and incubating at a temperature suitable for polymerization by the DNA polymerase to obtain a cTNA library; (iv) subjecting the cTNA library to a selection assay to obtain at least one or more selected cTNAs; and (v) incubating the one or more selected cTNAs with a primer, a SuperScript II reverse transcriptase, and dNTPs at a temperature suitable for SuperScript II reverse transcriptase activity to obtain a selected DNA template library.

In some embodiments, the diverse DNA template sequences are restricted to dA, dC, and dT. In some embodiments, the DNA template sequences contain 7-deaza-dGTP instead of dGTP.

TNAs can be selected from a cTNA library in step (iv) based on a number of different criteria and assays depending on a desired functionality or endpoint for the TNAs being generated. Accordingly, in some embodiments the selection assay in sep (iv) includes selection of one or more cTNAs from the cTNA library based on affinity for a ligand. Examples of suitable affinity assays known in the art include, but are not limited to, aptamer affinity chromatography, systematic evolution of ligands by exponential enrichment (SELEX), and kinetic capillary electrophoresis. In other embodiments, selection of one or more cTNAs from the cTNA library is based on a catalytic activity. Methods for assaying and selecting catalytic activities, e.g., ribozyme activities, are known in the art as described in, e.g., Link et al. (2007), Biol Chem 388(8):779-786. In some embodiments, one or more cTNAs are selected based on a desired fluorescence emission. See, e.g., Paige et al (2011), Science, 333(6042):642-646.

In the various methods described herein, hybridization between a primer and its target sequence is generally carried out under high stringency conditions under which the primer is annealed with its complementary template sequence at a temperature approximately 5° C. below the primer's melting temperature Tₘ.

Also described herein are TNA transcription systems. In various embodiments a TNA transcription system includes the following components: a single stranded DNA template, a DNA polymerase comprising an amino acid sequence at least 95% identical to the amino acid sequence of Therminator™ DNA polymerase, tTTP, tGTP, tATP; and (i) dCTP; or (ii) a combination of tCTP and dCTP.

Also disclosed herein are TNA reverse transcription systems. Generally a TNA reverse transcription system, as described herein, includes: a TNA template comprising dC, a SuperScript II reverse transcriptase, and dNTPs.

### EXAMPLES

### Example 1

### TNA Synthesis by Primer-Extension on a DNA Template

The DNA primer P1 was 5′-end labeled by incubation in the presence of [γ-32P] ATP with T4 polynucleotide kinase for 1 h at 37° C. The ³²P labeled primer was annealed to the DNA template (Table 1) in 1× ThermoPol buffer [20 mM Tris-HCl, 10 mM (NH₄)₂SO₄, 10 mM KCl, 2 mM MgSO₄, 0.1% Triton X-100, pH 8.8 at 25° C.] by heating at 95° C. for 5 min and cooling on ice. Primer extension reactions were performed in 10 μl volumes containing 100 μM tNTPs (or a combination of defined tNTP and dNTP mixtures), 500 nM primer-template complex, 1 mM DTT, 100 μg/ml BSA, 1.25 mM MnCl₂ and 0.1 U/μl Therminator DNA polymerase. Reactions were initiated by adding the tNTP substrates to a solution containing all other reagents and heating the mixture for 1 h at 55° C. Primer extension products were analyzed by 20% denaturing polyacrylamide gel electrophoresis, imaged with a phosphorimager, and quantified using ImageQuant software (GE Healthcare Biosciences, Pittsburgh, Pa.).

TABLE 1
Sequences of primers, templates and oligonucleotide substrates

| Name | Sequence |
|---|---|
| Primer P1 | 5′-GACACTCGTATGCAGTAGCC-3′(SEQ ID NO: 2) |
| Primer P2 | 5′-CTTTTAAGAACCGGACGAACGACACTCGTTTGCAGTAGCC-3′ |
| | (SEQ ID NO: 3) |
| Primer P3 | 5′-TGTCTACACGCAAGCTTACA-3′(SEQ ID NO: 4) |
| Primer P4 | 5′-CTTTTAAGAACCGGACGAAC-3′(SEQ ID NO: 5) |
| Template | 5′-TGTCTACACGCAAGCTTACACCATTCTTTAACAGTATCACTATATCCATT |
| 4NT.3G | TACGAGTCAACATTAACCTCGGCTACTGCATACGAGTGTCAAAAAAAAAA-3′ |
| | (SEQ ID NO: 6) |
| Template | 5′-TGTCTACACGCAAGCTTACATTAAGACTCGCCATGTTACGATCTGCCAAG |
| 4NT.9G | TACAGCCTTGAATCGTCACTGGCTACTGCATACGAGTGTCAAAAAAAAAA-3′ |
| | (SEQ ID NO: 7) |
| Template | 5′-TGTCTACACGCAAGCTTACATTAAGACTCGACATGATACGATCTGACAAG |
| 4NT.9GA | AACAGACTTGAATCGACACTGGCTACTGCATACGAGTGTCAAAAAAAAAA-3′ |
| | (SEQ ID NO: 8) |
| Template | 5′-TGTCTACACGCAAGCTTACAAACTCCATTACCTATTCAACTTACAATCCT |
| 3NT.ATC | ATCAACCTTATAATCCACTTGGCTACTGCATACGAGTGTCAAAAAAAAAA-3′ |
| | (SEQ ID NO: 9) |
| Substrate RNA | 5′-AAAAUUUAUUUAUUAA-3′(SEQ ID NO: 10) |
| S1 | |
| Substrate DNA | 5′-AAAATTTATTTATTAA-3′(SEQ ID NO: 11) |
| S2 | |
| Substrate TNA | 3′-AAAATTTATTTATTAA-2′(SEQ ID NO: 12) |
| S3 | |
| Substrate RNA | 5′-GGGAGGAGGAUUACCCCUCGUUAAUAAAUAAAUUUUCUCUCGUGAUCGG |
| S4 | GUAGCUGGACGCGACGGGUCC-3′(SEQ ID NO: 13) |

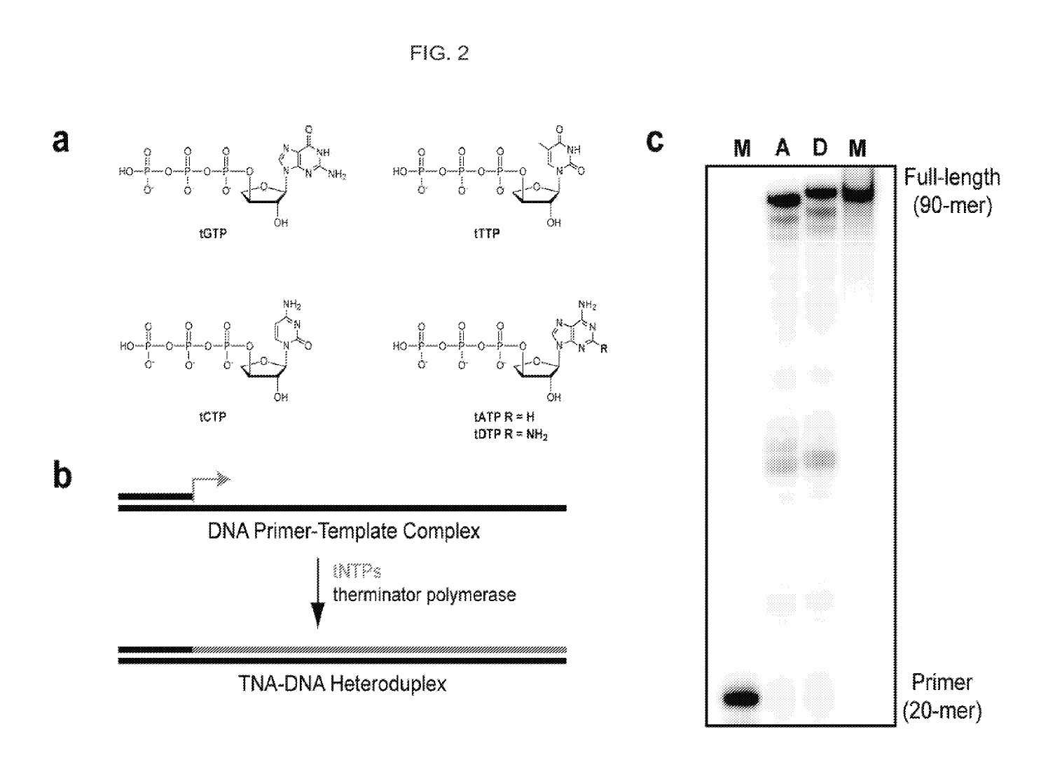

We began by chemically synthesizing each of the α-L-threofuranosyl nucleoside triphosphates (tNTPs) required for our study. This included TNA triphosphates with all four natural bases: tTTP, tATP, tCTP, and tGTP) as well as the diaminopurine analogue (tDTP) of adenine threofuranosyl 3′-triphosphate (FIG. 2a). Previous studies have established that the diaminopurine modification strongly enhances the thermodynamic stability of TNA/TNA, TNA/RNA, and TNA/DNA duplexes (for example, ΔΔG=4.7 kcal/mol, tD12/tT12 versus tA12/tT12). This modification also accelerates the rate of non-enzymatic template-directed ligation of TNA ligands and improves the efficiency of polymerase-mediated extension of tTTP residues on a DNA template. While our earlier work focused exclusively on the use of tDTP as substrate for TNA synthesis, we became concerned that the diaminopurine analogue might complicate the analysis of future TNA aptamers and enzymes. One could imagine that the presence of an additional proton donor group on the adenine base would make secondary structure prediction more difficult due to the enhanced potential for alternative non-Watson-Crick base pairing modes. A further concern is that structural differences between TNA and natural DNA and RNA are no longer limited to the sugar-phosphate backbone, which could obfuscate future comparisons made with previously evolved aptamers and enzymes.

To address these concerns, we examined the efficiency of tATP as a substrate for Therminator™ DNA polymerase. As illustrated in FIG. 2b, a synthetic DNA primer was annealed to a synthetic DNA library that contained a random region of 50-nts flanked on either side with a 20-nt primer binding site. Therminator DNA polymerase was challenged to extend the DNA primer with up to 70 sequential TNA residues to produce a library of TNA molecules containing either adenine or diaminopurine nucleotides in the product strands. Primer-extension assays were performed by incubating the polymerase for 1 hour at 55° C. in reaction buffer supplemented with 1.25 mM MnCl₂. We have previously shown that manganese ions dramatically enhance the efficiency of TNA synthesis. Analysis of the extension products by denaturing polyacrylamide gel electrophoresis reveals that tATP and tDTP are equally efficient substrates for Therminator™ DNA polymerase. In both cases, the DNA primer was completely extended with TNA residues to make the desired full length product (FIG. 2c). While we had previously constructed TNA libraries with diaminopurine residues, this was the first demonstration where a TNA library was prepared using all four natural nucleobases. Since no difference in the amount of full-length product was observed between the two sets of in vitro transcription reactions, we concluded that tATP is an efficient substrate for Therminator™ DNA polymerase in the enzyme-mediated polymerization of TNA.

### Example 2

### In Vitro Reverse Transcription of TNA into DNA

The ³²P-labelled DNA primer P3 was annealed to a TNA template in 1× First Strand buffer [50 mM Tris-HCl, 75 mM KCl, 3 mM MgCl₂ (pH 8.3 at 25° C.)] by heating at 95° C. for 3 min and cooling on ice. Primer extension reactions contained 500 μM dNTPs, 100 nM primer-template complex, 10 mM DTT, 3 mM MgCl₂, 1.5 mM MnCl₂ and 10 U/μl SuperScript II reverse transcriptase. Reactions were initiated by adding the enzyme to a solution containing all other reagents, and heating the reaction mixture for 1 h at 42° C. Primer extension products were analyzed by 20% denaturing polyacrylamide gel electrophoresis, imaged with a phosphorimager, and quantified using ImageQuant software (GE Healthcare Biosciences, Pittsburgh, Pa.).

In order to generate a sufficient amount of TNA template to be used in a reverse transcription reaction, TNA synthesis reactions were performed as described above in Example 1 using unlabeled DNA primer P2 in a 400 μl reaction. After incubation for 1 hour at 55° C., the TNA product was separated from the DNA template by 10% denaturing polyacrylamide gel electrophoresis. The band corresponding to the TNA product was excised and the gel slices were electroeluted for 2 hours at 200V. The final solution was ethanol precipitated and quantified by UV absorbance.

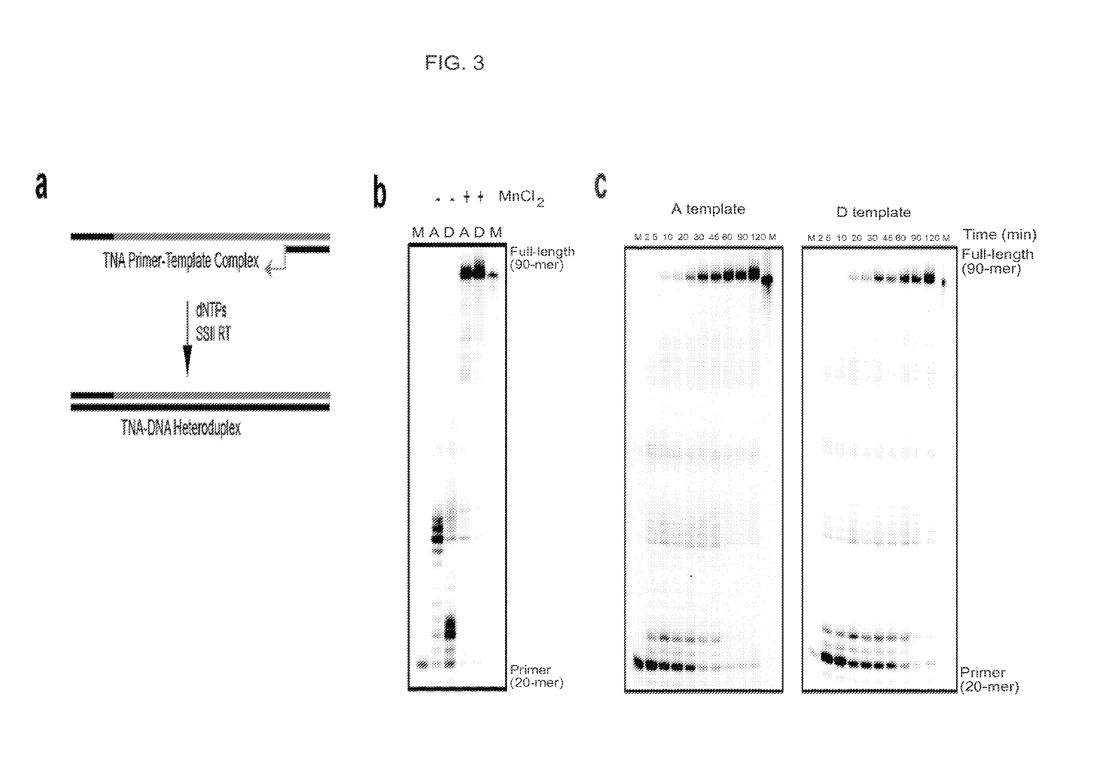

³²P-labelled DNA primer P3 was annealed to the TNA template in 1× First Strand buffer [50 mM Tris-HCl, 75 mM KCl, 3 mM MgCl₂ (pH 8.3 at 25° C.)] by heating at 85° C. for 3 min and cooling on ice. Primer extension reactions contained 500 μM dNTPs, 100 nM primer-template complex, 10 mM DTT, 3 mM MgCl₂, 1.5 mM MnCl₂ and 10 U/μ1 SuperScript II™ reverse transcriptase. Reactions were initiated by adding the enzyme to a solution containing all other reagents, and heating the reaction mixture for 1 h at 42° C. Primer extension products were analyzed by 20% denaturing polyacrylamide gel electrophoresis, imaged with a phosphorimager, and quantified using ImageQuant software (GE Healthcare Biosciences, Pittsburgh, Pa.). As shown in FIG. 3(b), reverse transcription of TNA was strongly dependent on the presence of Mn²⁺, and as shown in FIG. 3(c), full length cDNA was synthesized from both adenosine (A) and diaminopurine (D)-containing TNA templates over a period of 20-120 minutes.

The in vitro selection of XNA molecules in the laboratory requires enzymes that can transcribe and reverse transcribe XNA polymers with high efficiency and fidelity. In a recent new advance, Pinheiro et al. used a compartmentalized self-tagging strategy to evolve several polymerases with XNA activity. One of these enzymes, RT521, was created from TgoT, a variant of the replicative polymerase from Thermococcus gorgonarius, for the ability to reverse transcribe HNA back into DNA. In addition to HNA reverse transcriptase activity, RT521 was also found to reverse transcribe other XNA polymers with varying degrees of efficiency. This included arabinonucleic acids, 2′-fluoro-arabinonucleic acids and TNA35. The observation that RT521 could reverse transcribe portions of a TNA template into DNA led us to consider this enzyme as a possible polymerase for the replication TNA polymers in vitro.

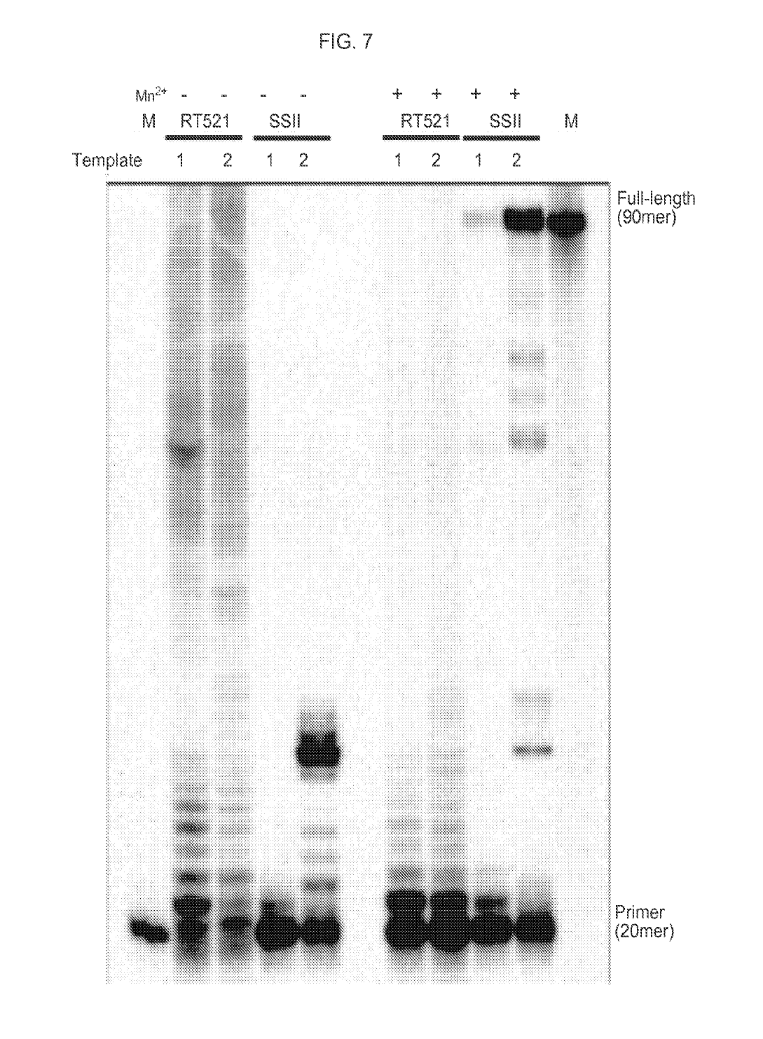

To examine the activity of RT521 as a TNA-dependent DNA polymerase, we performed a polymerase activity assay to access the ability for RT521 to reverse transcribe long TNA templates into DNA. Because it is not possible to generate long TNA polymers by solid-phase synthesis, we transcribed a DNA template into TNA using Therminator™ DNA polymerase (FIG. 3a). The resulting TNA polymer was purified by denaturing polyacrylamide gel electrophoresis and used as a template for reverse transcription. A second DNA primer was then annealed to the 2′-end of the TNA strand and reverse transcription was attempted by incubating the primer-template complex with RT521 for 24 hours at 65° C. Although some variation was observed among the different TNA templates, the best primer-extension reaction produced full-length products that were barely detectable by polyacrylamide gel electrophoresis (FIG. 7).

In an attempt to improve the efficiency of TNA-dependent DNA polymerization by RT521, we explored a variety of conditions that have proven helpful in the past. To our surprise, varying the reaction time, salt conditions, and enzyme concentration all proved ineffective. Even the addition of manganese ions, which is known to relax the specificity of many DNA polymerases, inhibited the reaction. The presence of diaminopurine residues in the TNA template also failed to improve the yield of full-length product. The limited TNA synthesis observed in these reactions may reflect an unknown sequence specificity of the enzyme. Alternatively, it is also possible that the sample of RT521 used in our study was less active than the sample used in the original study by Pinheiro et al.

However, close examination of the previous reverse transcription reaction revealed a substantial amount of truncated product, suggesting that RT521 may require further optimization before it can function as an efficient TNA-dependent DNA polymerase.

Recognizing the limitations of RT521, we pursued other enzymes as possible candidates for a TNA reverse transcriptase. In this regard, we have previously screened a wide range of natural and mutant DNA and RNA polymerases for the ability to copy a short chimeric DNA-TNA template containing nine TNA residues in the template region. This study identified the reverse transcriptases MMLV and SuperScript II (SSII) as efficient TNA-dependent DNA polymerases that could copy a short TNA template into DNA with ˜30% full-length product conversion observed after an incubation of 1 hour at 42° C. To determine whether these enzymes could be made to function on longer TNA templates, we explored a range of conditions that would allow the enzymes to copy a 90-nt TNA template back into DNA. Since it was possible that diaminopurine would enhance the efficiency of reverse transcription, we performed the polymerase activity assay on in vitro transcribed TNA containing either adenine or diaminopurine nucleotides in the template strand. Preliminary studies indicated that SSII functioned with greater efficiency and reproducibility than MMLV. Subsequent optimization of this reaction led us to discover conditions that enabled SSII to reverse transcribe the entire TNA template into DNA (FIG. 3b). Optimal extension was observed using new enzyme and a reaction buffer that contained a freshly prepared solution of 1.5 mM MnCl₂. Under these conditions, the adenine- and diaminopurine-containing TNA templates were efficiently reverse transcribed back into DNA. In the absence of MnCl₂, the reaction was significantly impeded with SSII terminating reverse transcription early into the primer extension process.

To assess the efficiency of SSII-mediated reverse transcription, we performed a time course analysis to compare the rate of product formation as a function of template composition. Analysis of product formation over time revealed that reverse transcription of the adenine-containing template is complete in 1 hour, while the diaminopurine-containing template required nearly 2 hours to copy the TNA template into DNA (FIG. 3c). The higher efficiency of the adenine-containing template further supports the use of tATP as a substrate for TNA synthesis. Taken together, the transcription and reverse transcription results demonstrate that commercial enzymes can be made to replicate TNA polymers with high efficiency, which is remarkable considering the significant structural differences between the threofuranosyl and (deoxy)ribofuranosyl backbones of TNA and DNA (or RNA), respectively.

### Example 3

### Fidelity Assay

DNA sequencing was used to measure the fidelity for the overall process of TNA replication and cloning. DNA templates of a defined sequence were transcribed into TNA as described above using primer P2. Primer P2 has an internal reference nucleotide that is designed to unambiguously distinguish cDNA obtained from TNA replication from the starting DNA template. The DNA-TNA heteropolymer was purified by denaturing polyacrylamide gel electrophoresis, and reverse transcribed back into DNA. The resulting cDNA strand was amplified by PCR using primers that matched the outside region of P2 (i.e. P3 and P4). AccuPrime Taq High Fidelity DNA Polymerase was used to minimize possible mutations caused by PCR. Additionally, separate PCR reactions were performed on purified TNA templates to confirm that the PCR product was amplified from cDNA generated in TNA reverse transcription. PCR products were cloned into pJET1.2 vector, transformed into E. coli XL1-Blue competent cells, grown to log phase, the vector was isolated using PureYield™ Plasmid Miniprep System (Promega, Madison, Wis.). Isolated vectors were sequenced at the ASU DNA Sequencing Facility.

We measured the fidelity of TNA replication by sequencing the cDNA product of the reverse transcription reaction after amplification by PCR. This fidelity assay measures the aggregate fidelity of a complete replication cycle (DNA→TNA→DNA), which is operationally different than the more restricted view of fidelity as the accuracy of a single-nucleotide incorporation event. The fidelity determined by this assay is the actual accuracy with which full-length TNA is synthesized and reverse transcribed, and therefore reflects the combined effects of nucleotide misincorporation, insertions and deletions (indel), and any mutations that occur during PCR amplification and cloning.

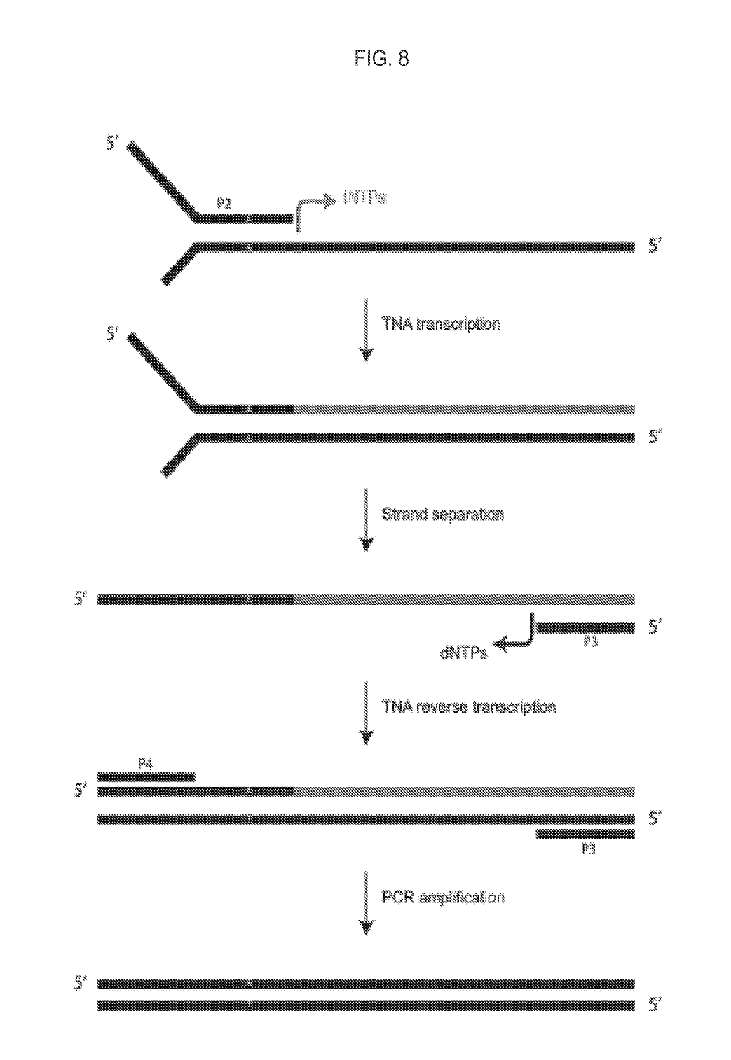

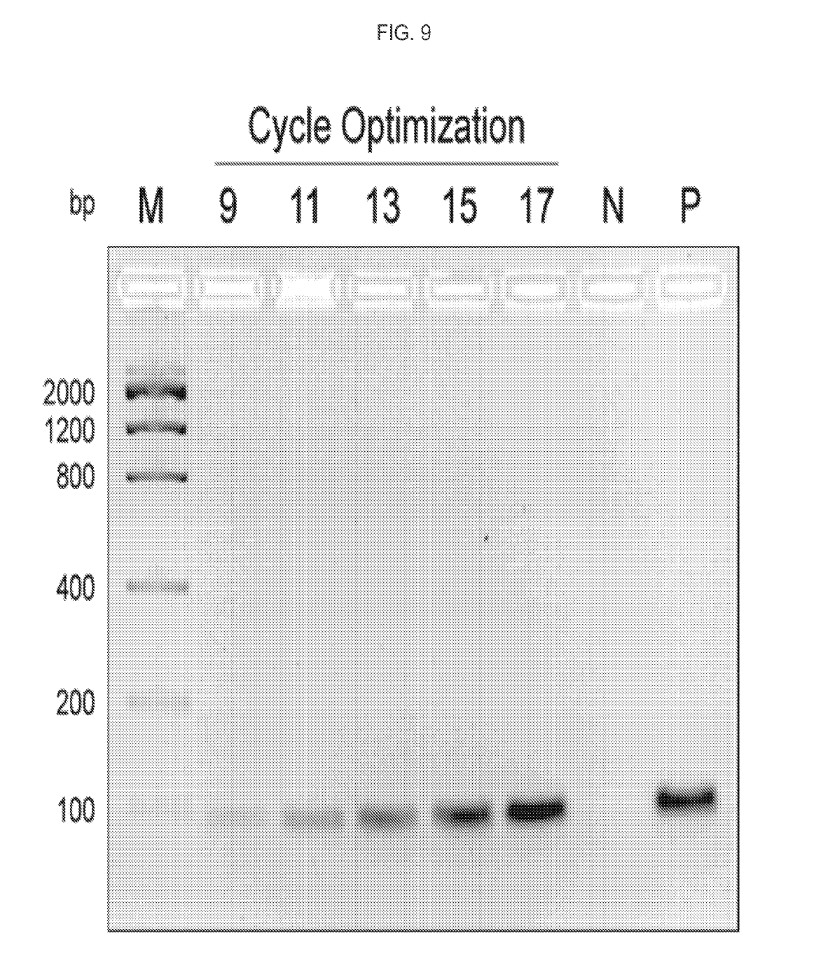

Several controls were implemented to ensure that the sequencing results represented the true fidelity of TNA replication (FIG. 8). First, to eliminate any possibility of contamination by the starting DNA template, the DNA primer-template complex used for TNA transcription was partially unpaired and contained additional nucleotides in the primer strand to facilitate separation of the TNA product by denaturing polyacrylamide gel electrophoresis. Second, all PCR amplification steps were performed using a negative control that contained the purified TNA template prior to reverse transcription. In no cases did we observe a DNA band in this lane, demonstrating that the purification step effectively separated the TNA transcript from the DNA template (FIG. 9). Third, to unambiguously demonstrate that each DNA sequence derived from a complete cycle of TNA replication, the DNA primer used for TNA transcription was engineered to contain a single-nucleotide mismatch that resulted an A→T transversion in the sequenced product. These controls allowed us to determine the actual fidelity of TNA replication with confidence.

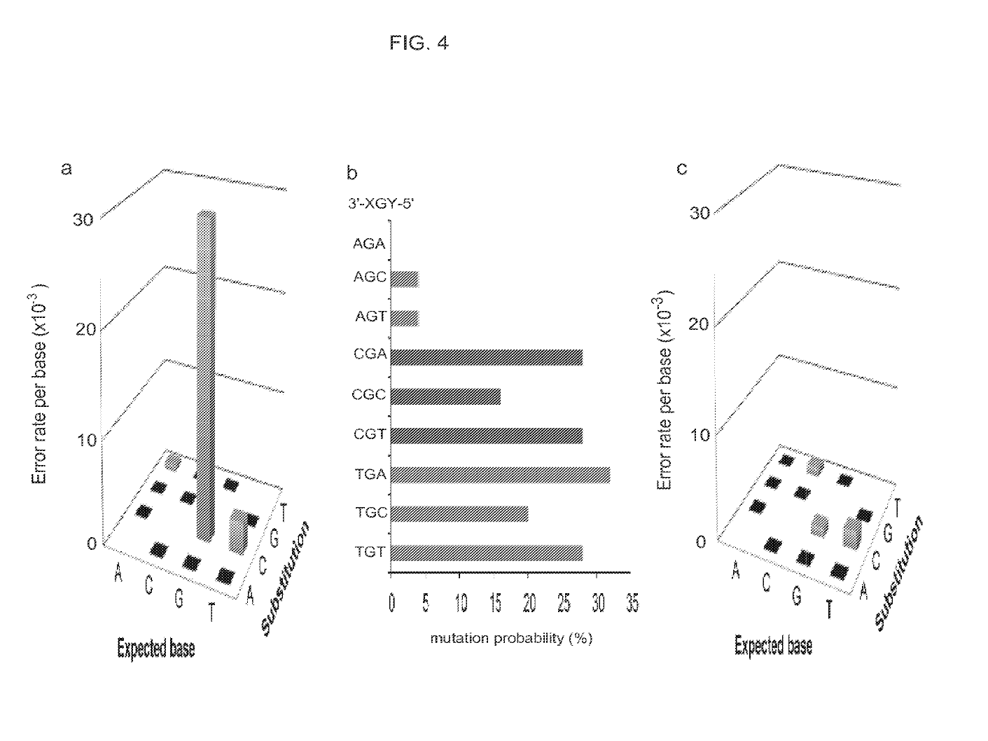

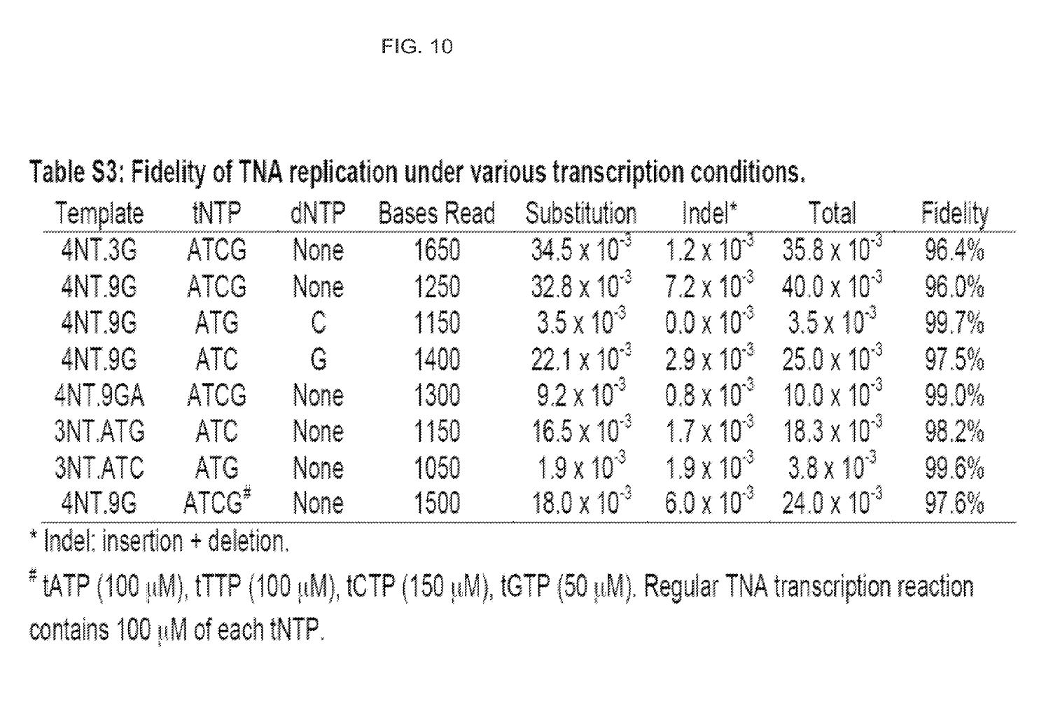

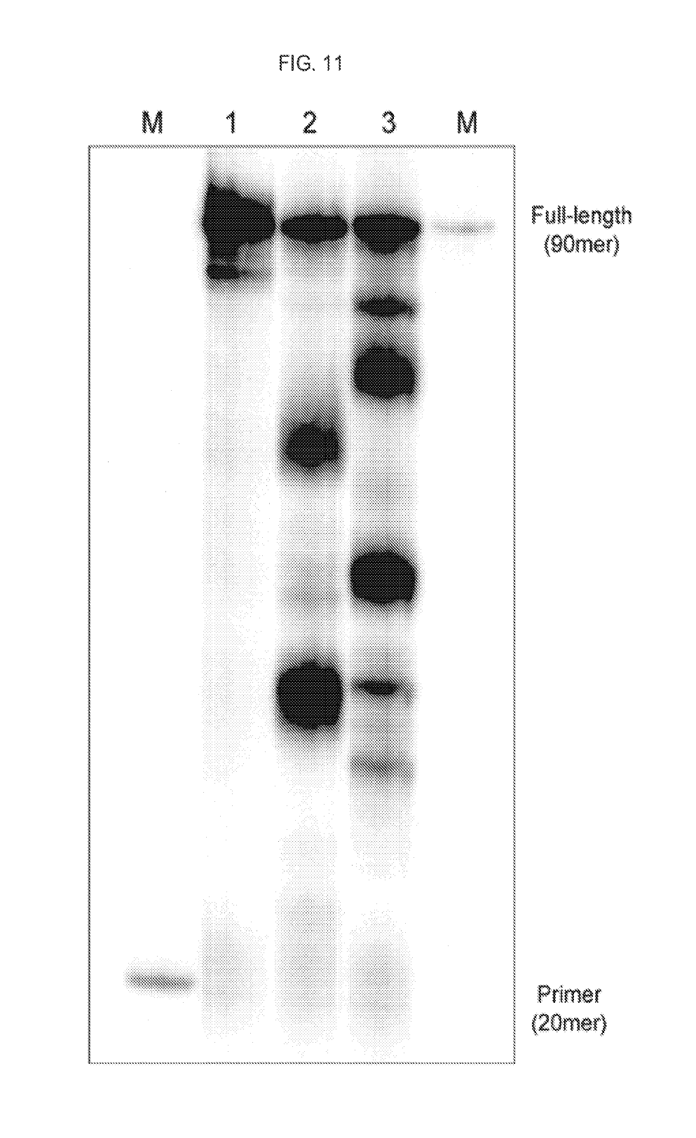

We began by measuring the fidelity of TNA replication for the adenine-containing template used in the reverse transcription assay with SSII. This template, referred to as 4NT.3G, derives from a single sequence that was present in the L3 library30. The L3 library was designed to overcome the problem of polymerase stalling at G-repeats by reducing the occurrence of G residues in the template to 50% the occurrence of A, C, and T. Our earlier work on TNA transcription established the L3 library as an efficient design strategy for generating pools of full-length TNA molecules. While TNA replication on 4NT.3G resulted in an overall fidelity that was comparable with other XNA replication systems (96.4%), detailed analysis of the mutation profile indicated that G→C transversions account for 90% of the genetic changes (FIG. 4a; FIG. 10). Since iterative replication cycles of the L3 library would eventually bias TNA replication toward a population of DNA sequences that were overly enriched in cytidine residues, we decided to ascertain the propensity for mutagenesis by examining the role of nearest-neighbor effects in the DNA template. We designed a synthetic DNA template (4NT.9G) containing all of the possible combinations of A, C, and T nucleotides on the 3′ and 5′ side of a central G residue. We avoided the triplets NGG, GGN, and GGG due to their ability to terminate primer extension (for example, see FIG. 11). We found that the frequency of a G→C transversion is ˜25% when a pyrimidine (C or T) precedes G in the template, but only ˜3% when G is preceded by A (FIG. 4b). No correlation was observed between the identity of the 5′ nucleotide residue and the frequency of transversion, suggesting that mutagenesis occurs during the transcription step of TNA replication. We tested this hypothesis by repeating the triplet fidelity study using a nucleotide mixture in which the tCTP substrate was replaced with dCTP. Under these conditions, mutagenesis is suppressed and the overall fidelity of TNA replication increases to 99.6% (FIG. 4c; FIG. 10).

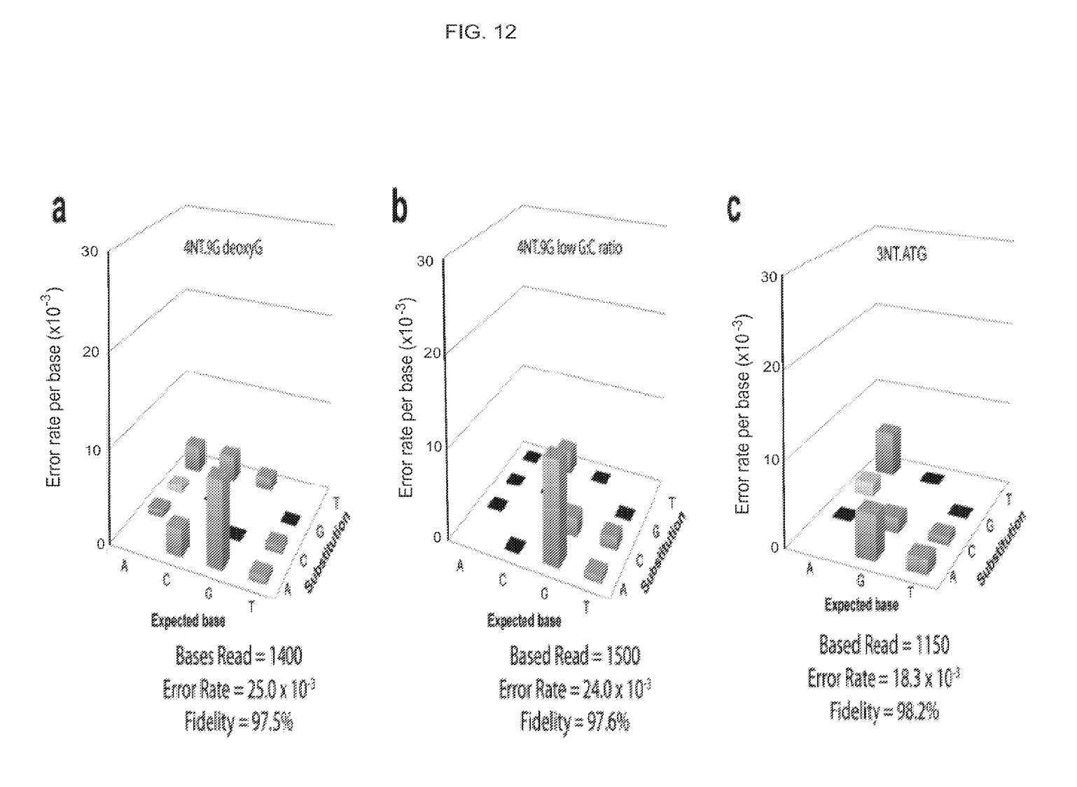

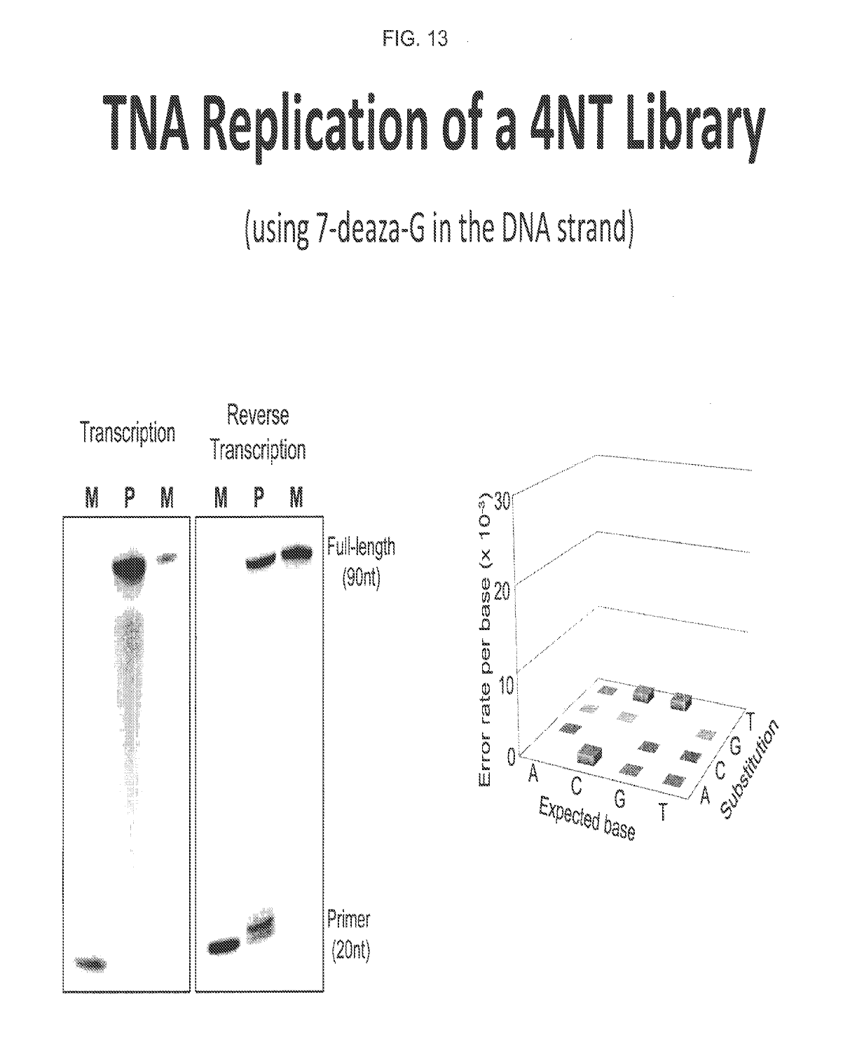

While the precise molecular details of the G→C transversion remain unknown, our results suggest that base stacking plays an important role in the misincorporation of tGTP opposite deoxyG in the template. This prediction is supported by the fact that the frequency of dG:tG mispairing increases 10-fold when G-nucleotides in the template are preceded by pyrimidine residues, indicating that purine residues (A or G) on the growing TNA strand stabilize the incoming tGTP substrate via base stacking interactions. To better understand the problem of dG:tG mispairing, we measured the fidelity of TNA replication using different combinations of template and substrate (FIG. 12). Biasing the nucleotide mixture with lower amounts of tGTP and higher amounts of tCTP increased the fidelity to 97.6% and reduced the problem of G→C transversions.

Substituting tGTP for dGTP and assaying a template devoid of C residues produced similar results with 97.5% and 98.2% fidelity, respectively. The mutational profiles obtained under these conditions provide evidence that dG:tG mispairing can be overcome by engineering DNA templates to avoid the problem of nucleotide misincorporation.

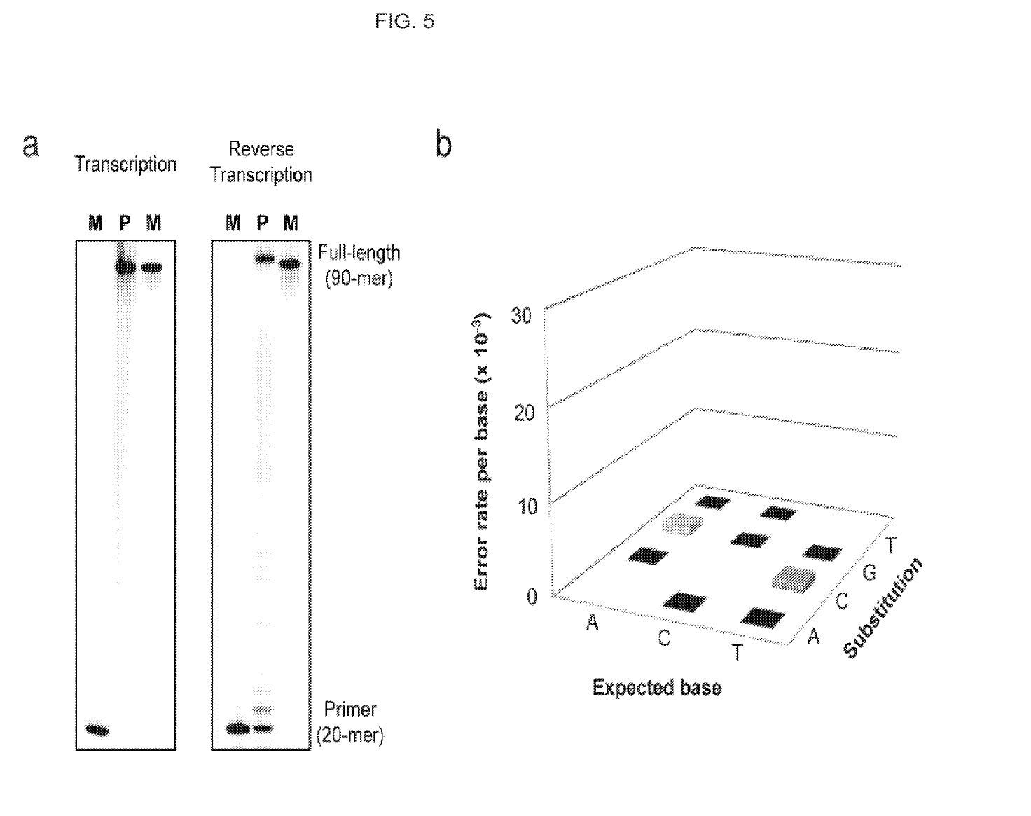

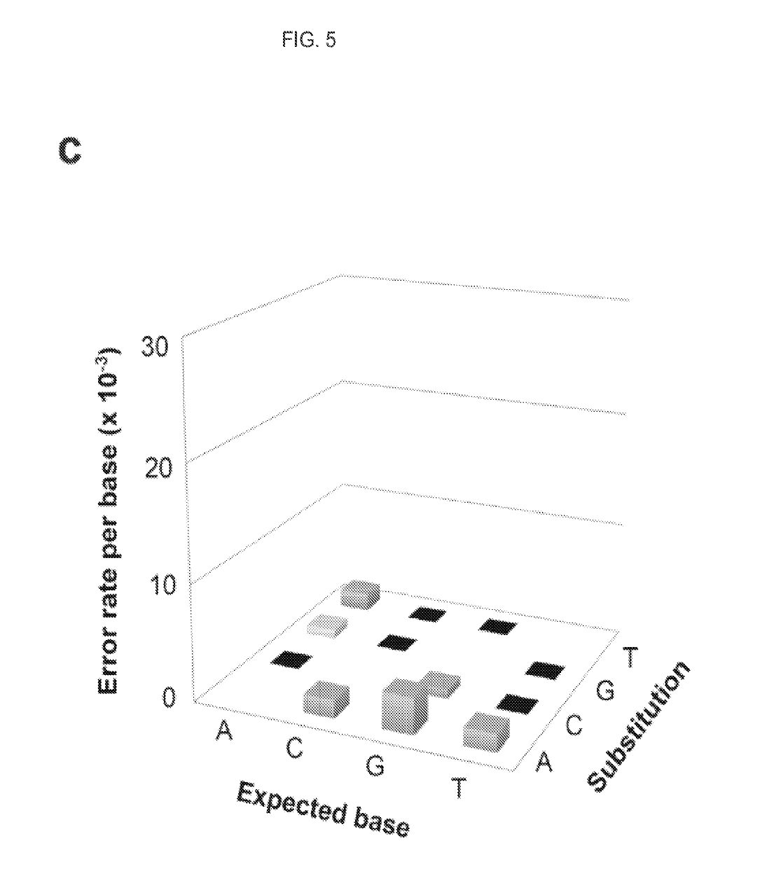

In an effort to further improve the fidelity of TNA replication, we examined the mutational profile of two different types of DNA templates that were designed for high fidelity replication. The first template, 3NT.ATC, contained a central region of 50-nts that was composed of a random distribution of A, T, and C residues that were flanked by two 20-nt fixed-sequence primer-binding sites. This sequence derived from library L2, which we used previously to evolve a TNA aptamer to human thrombin. We found that the L2 library transcribes and reverse transcribes with very high efficiency as judged by the amount of starting primer that is extended to full-length TNA product and the absence of any significant truncated products (FIG. 5a). Consistent with the efficient replication of the L2 library, the template 3NT.ATC exhibits an overall fidelity of replication of 99.6% (FIG. 5b), which is similar to the fidelity of in vitro RNA replication. Similar results (99.0% fidelity) were obtained with a four-nucleotide sequence, 4NT.9GA, which is identical to the DNA template 4NT.9G, except that each of the nine G residues in the template was preceded by an adenine nucleotide to minimize dG:tG mispairing in the enzyme active site (FIG. 5c). These results demonstrate that commercial enzymes are capable of replicating TNA with high efficiency and fidelity, both of which are essential for future in vitro selection experiments.

It is hypothesized that dG:tG mispairing occurs through a Hoogsteen base pairing mode rather than the traditional Watson-Crick mode. Nitrogen-7 is critically important for the formation of the Hoogsteen base pair and removal of nitrogen-7 from guanosine in either the templating G residues or substrate tGTPs would eliminate that base pairing mode and prevent dG:tG mispairing. The 4NT.9G template and an unbiased four nucleotide library containing equal amounts of dC, dG, dA, and dT containing 7-deaza-dG in place of dG were generated by asymmetric PCR. The 7-deaza-dG asymmetric PCR reaction is identical to normal asymmetric PCR reactions aside from dGTP being replaced by 7-deaza-dGTP in an equal concentration. PCR reaction products were purified by polyacrylamide gel electrophoresis and used in TNA extension assays and fidelity measurements identical to above. We found that the replication fidelity of four nucleotide templates improved from 96.4% to 99.6%. The fidelity was on par with templates containing only dA, dT, and dC. Additionally, TNA transcription of the four nucleotide library measured by primer extension and gel electrophoresis showed equivalent full length product to three nucleotide libraries.

### Example 4

### Nuclease Stability Assay

DNA, RNA, and TNA oligonucleotide substrates (1 nmol) were incubated for up to 72 hours at 37° C. in presence of RQ1 DNase or RNase A using the manufacture's recommended conditions. The DNase reaction contained 1× RQ1 DNase reaction buffer [40 mM Tris-HCl, 10 mM MgSO₄, 1 mM CaCl₂, pH 8.0] and 0.2 U/μ1 of RQ1 RNase-free DNase in reaction volume of 10 μl. The RNase reaction contained 50 mM NaOAc (pH 5.0) and 0.24 μg/μl RNase A in a reaction volume of 10 μl. Time course reactions were performed by initiating multiple reactions in parallel, removing individual tubes at defined time points, quenching the reaction by the addition of 7 M urea and 20 mM EDTA, storing the quenched reactions at −20° C. until the time course was complete. Time-dependent oligonucleotide stability against DNase or RNase was analyzed by 20% denaturing polyacrylamide gel electrophoresis, and visualized by UV shadowing.

RNA template T1 was synthesized by in vitro transcription using T7 RNA polymerase. After purification by denaturing PAGE, the RNA transcript was dephosphorylated using calf intestinal alkaline phosphatase, and then 5′-end labeled by incubation in the presence of [γ-³²P] ATP with T4 polynucleotide kinase. ³²P-labeled RNA template T1 (25 pmol) was incubated with a complementary DNA oligonucleotide probe S2 or TNA oligonucleotide probe S3 (50 pmol) for 15 min at 37° C. Each reaction contained 44 μl of reaction buffer [10 mM Tris-HCl, 25 mM KCl, 1 mM NaCl, and 0.5 mM MgCl₂, pH 7.5] and 6 μl RNase H (5 U/μl). Control tubes received buffer in place of enzyme. Aliquots were removed at the indicated time points, quenched by the addition of 7 M urea and 20 mM EDTA, and analyzed by 20% denaturing polyacrylamide gelelectrophoresis.

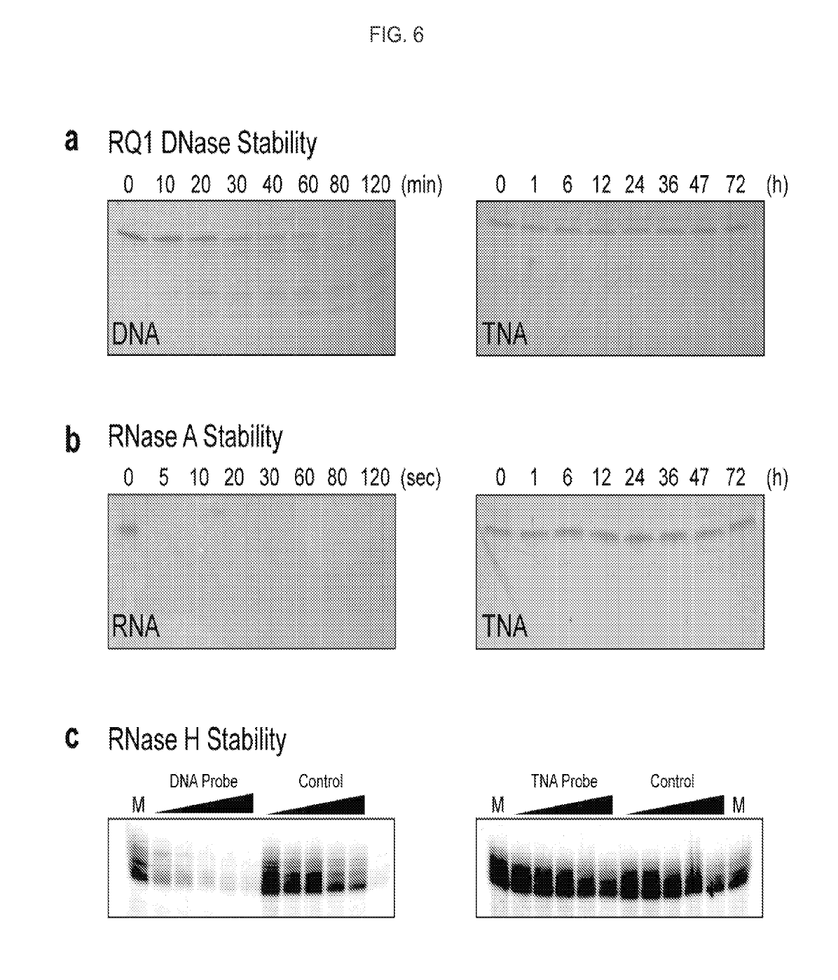

A major goal of synthetic genetics is to create nuclease resistant aptamers and enzymes that function in complex biological environments. To evaluate the nuclease stability of TNA, we synthesized a synthetic TNA 16-mer having the sequence 3′-AAAATTTATTTATTAA-2′ (SEQ ID NO:14) by solid phase phosphoramidite chemistry. The TNA oligonucleotide was tested for nuclease stability against the enzymes RQ1 DNase and RNase A, which degrade DNA and RNA, respectively. In both cases, 1 nmol of the TNA sample was incubated at 37° C. in a reaction buffer of 40 mM Tris-HCl, 10 mM MgSO₄, 1 mM CaCl₂ (pH 8.0) for the DNase digestion and a reaction buffer of 50 mM NaOAc (pH 5.0) for the RNase digestion. The samples were removed at specified time points, quenched with urea, and analyzed by denaturing polyacrylamide gel electrophoresis. As a control, synthetic DNA and RNA strands with the same sequence were incubated with their respective nuclease and analyzed under time frames that coincided with their degradation. As expected, the DNA sample is rapidly degraded in the presence of RQ1 DNase and exhibited a half-life of ˜30 minutes (FIG. 6a). The case was even more extreme for the RNA sample, which degraded in a matter of seconds and exhibited a half-life of <10 seconds (FIG. 6b). In contrast to the natural DNA and RNA samples, the TNA sample remained undigested even after 72 hours in the presence of pure nuclease (FIG. 6a,b). This result demonstrates that enzymes that degrade DNA and RNA do not easily recognize the threofuranosyl backbone of TNA. Antisense oligonucleotides are widely used to alter intracellular gene expression patterns by activating RNase H activity. RNase H is an endoribonuclease that specifically hydrolyzes the phosphodiester bonds of RNA in DNA-RNA duplexes to produce 3′ hydroxyl and 5′ monophosphate products. Given the importance of alternative nucleic acid structures as antisense therapeutics, we felt that it would be interesting to examine the recognition properties of TNA-RNA hybrids by RNase H. We hybridized a 16-mer TNA oligonucleotide to the target site of a 70-mer synthetic RNA strand produced by in vitro transcription. To establish a positive control for RNase H activity, the analogous 16-mer DNA probe was hybridized to the RNA target. The DNA and TNA samples were incubated at 37° C. in the presence and absence of the enzyme in buffer containing 10 mM Tris-HCl, 25 mM KCl, 1 mM NaCl, and 0.5 mM MgCl₂ (pH 7.5). Samples were removed at specified time points, quenched with urea, and analyzed by denaturing polyacrylamide gel electrophoresis. As expected, the DNA-RNA hybrid is rapidly degraded (half-life <1 minute) in the presence of RNase H, while the TNA-RNA hybrid remained intact even after an incubation of 16.5 hours indicating that TNA does not promote RNase H activity in vitro (FIG. 6c).

The invention has been described in connection with what are presently considered to be the most practical and preferred embodiments. However, the present invention has been presented by way of illustration and is not intended to be limited to the disclosed embodiments. Accordingly, those skilled in the art will realize that the invention is intended to encompass all modifications and alternative arrangements within the spirit and scope of the invention as set forth in the appended claims.

What is claimed is:

1. A method for synthesizing a threose nucleic acid polymer, comprising:
contacting a single stranded DNA template hybridized to a primer with a DNA polymerase comprising an amino acid sequence at least 95% identical to the amino acid sequence of SEQ ID NO:1 in the presence of tTTP, tGTP, tATP; and (i) dCTP; or (ii) a combination of tCTP and dCTP; and incubating at a temperature suitable for polymerization by the DNA polymerase to obtain a threose nucleic acid.

2. The method of claim 1, wherein the DNA polymerase is in the presence of tTTP, tGTP, tATP, and dCTP.

3. The method of claim 2, wherein the contacting step is done in the substantial absence of tCTP.

4. A threose nucleic acid generated by the method of claim 3

5. The method of claim 1, wherein the DNA polymerase is in the presence of tATP, tTTP, tGTP, and a combination of tCTP and dCTP.

6. The method of claim 1, wherein the DNA polymerase comprises the amino acid sequence of SEQ ID NO:1.

7. The method of claim 1, wherein the single stranded DNA template sequence is restricted to the nucleotides dA, dC, and dT.

8. The method of claim 1, wherein the single stranded DNA template sequence comprises 7-deaza-dGTP instead of dGTP.

9. A method for reverse transcribing a threose nucleic acid, comprising contacting a threose nucleic acid template with a SuperScript II reverse transcriptase in the presence of a primer and dNTPs, dNTP analogs, or a combination thereof to obtain a threose nucleic acid reverse-transcription mix, and incubating the mix at a temperature suitable for SuperScript II reverse transcriptase activity to obtain a cDNA copy of the threose nucleic acid template, wherein the threose nucleic acid template comprises deoxycytidine.

10. A method for molecular evolution of threose nucleic acids, the method comprising:
(i) providing a DNA template library comprising diverse DNA template sequences;
(ii) hybridizing the template library with one or more complementary primer sequences;
(iii) incubating the hybridized template library with a DNA polymerase comprising an amino acid sequence at least 95% identical to the amino acid sequence of SEQ ID NO:1 in the presence of tTTP, tGTP, tATP; and (i) dCTP; or (ii) a combination of tCTP and dCTP; and incubating at temperature suitable for polymerization by the DNA polymerase to obtain a cTNA library;
(iv) subjecting the cTNA library to a selection assay to obtain at least one or more selected cTNAs; and
(v) incubating the one or more selected cTNAs with a primer, a SuperScript II reverse transcriptase, and dNTPs at a temperature suitable for SuperScript II reverse transcriptase activity to obtain to obtain a selected DNA template library.

11. The method of claim 10, wherein the diverse DNA template sequences are restricted to the nucleotides dA, dC, and dT.

12. The method of claim 10, wherein the selection assay in step (iv) comprises selection of one or more cTNAs based on affinity for a ligand.

13. The method of claim 10, wherein the selection assay in step (iv) comprises selection of one or more cTNAs based on a catalytic activity.

14. The method of claim 10, wherein the selection assay in step (iv) comprises selection of one or more cTNAs based on fluorescence emission.

15. The method of claim 10, wherein step (iii) is done in the substantial absence of tCTP.

16. The method of claim 10, wherein the DNA template library comprises DNA templates comprising 7-deaza-dGTP instead of dGTP.

17. A TNA transcription system comprising a single stranded DNA template, a DNA polymerase comprising an amino acid sequence at least 95% identical to the amino acid sequence of SEQ ID NO:1, tTTP, tGTP, tATP; and (i) dCTP; or (ii) a combination of tCTP and dCTP.

18. The TNA transcription system of claim 17, wherein the TNA transcription system comprises dCTP, but is substantially free of tCTP.

19. The TNA transcription system of claim 17, wherein the single stranded DNA template comprises 7-deaza-dGTP instead of dGTP.

20. A TNA reverse transcription system comprising a TNA template, a SuperScript II reverse transcriptase, and dNTPs; wherein the TNA template comprises dC.
